(19) United States
(12) Patent Application Publication
(10) Pub. No.: US 2026/0345434 A1
(43) Pub. Date: Nov. 5, 2026

(54) Stimulation of brain waste clearance

(71) Applicant: Deep Sleep Technologies B.V., Amsterdam (NL)

(72) Inventor: Lucia Maddalena Talamini, Amsterdam (NL)

(21) Appl. No.: 19/194,962

(22) Filed: Apr. 30, 2025

(51) Int. Cl. A61H 23/02; A61B 5/374

(52) U.S. Cl. CPC A61H 23/0236; A61B 5/374; A61H 2205/02; A61H 2230/105

(57) ABSTRACT

Systems and methods are described for stimulating glymphatic activity in a subject. The method comprises receiving data representative of brain activity of the subject, and executing a fitting algorithm to determine, based on the received data, a fitted periodic function. The fitted periodic function has a frequency, which frequency is generally one of one or more fitting parameters. The method further comprises determining that the frequency in a slow-oscillation range, and predicting a future point in time at which the brain activity of the subject exhibits a predetermined pattern. This prediction is based on the fitted periodic function. The method further comprises determining that the future point in time is within a search window. The method further comprises controlling a stimulus generator to generate a stimulus such that it is processed by the subject in an event time window comprising the future point in time.

### TECHNICAL FIELD

This disclosure relates to methods and systems for stimulating brain waste clearance, and in particular, though not exclusively, to methods and systems for stimulating brain waste clearance by stimulating slow oscillations of a sleeping subject, and to a computer program product enabling a computer system to perform such methods.

### BACKGROUND

Brain clearance, or brain waste clearance, is formally defined as the removal of metabolic waste products from neural tissues. Brain waste clearance can take place through several physiological pathways, including extracellular enzymatic degradation, cellular uptake in neuronal and non-neuronal cells, efflux into blood circulation via active transport across the blood-brain barrier, microglia-mediated phagocytosis, and clearance through the glymphatic system.

The glymphatic system is a brain waste clearance system that consists of perivascular pathways surrounding cerebral arteries and veins. The term ‘glymphatic’ reflects the dependence of this system on glial cells (e.g., astrocytes) that perform ‘lymphatic’ clearing of the brain. Driven predominantly by arterial pulsations, cerebrospinal fluid (CSF) flows from arterial perivascular spaces into the interstitial compartment of the brain via water channel proteins, called aquaporin-4. These proteins, located on the end feet of astrocytes, facilitate the exchange of CSF with the interstitial fluid (ISF). Following this exchange, fluids drain into the venous perivascular spaces and exit the brain through meningeal nodes, ultimately clearing it of excess fluids, cellular debris, neuronal metabolites, and neurotoxic proteins.

The glymphatic system is known to be most active during deep sleep, in particular so-called slow-wave sleep (SWS). Consequently, sleep disorders lead to an increased risk of poor brain waste clearance. Moreover, advanced age is associated with less deep sleep, again leading to an increased risk of poor brain waste clearance. Over time, poor brain waste clearance can lead to undue accumulation of brain waste substances, such as amyloid-β, phosphorylated tau, and a-synuclein. In turn, such accumulation of brain waste substances is associated with several (neurodegenerative) diseases, including Alzheimer's disease and Parkinson's disease.

Van Hattem et al., ‘Targeting Sleep Physiology to Modulate Glymphatic Brain Clearance’, Physiology 40:3 (May 2025) describes various possible approaches to target sleep physiology to modulate glymphatic brain clearance, including behavioural changes and pharmacological interventions.

Van den Bulcke et al., ‘Acoustic stimulation as a promising technique to enhance slow-wave sleep in Alzheimer's disease: results of a pilot study’, Journal of Clinical Sleep Medicine 19:12 (December 2023) describes using a commercially available headband device, known as the DREEM 2, on 7 patients with mild or moderate dementia. However, this study showed poor results; in particular, very few stimuli were provided (for example, in more than half of the nights in which brain stimulation was intended, no stimulation was given at all).

There is therefore a need in the art for a device and method for stimulating brain waste clearance.

### SUMMARY

It is an aim of embodiments in this disclosure to provide a system and method for stimulating brain waste clearance that avoids, or at least reduces the drawbacks of the prior art.

In an aspect, this disclosure relates to a method for stimulating glymphatic activity in a subject. The method comprises receiving data representative of brain activity of the subject, and executing a fitting algorithm to determine, based on the received data, a fitted periodic function. The fitted periodic function has a frequency, which frequency is generally one of one or more fitting parameters. The method further comprises determining that the frequency is in a slow-oscillation range, and predicting a future point in time at which the brain activity of the subject exhibits a predetermined pattern. This prediction is based on the fitted periodic function. The method further comprises determining that the future point in time is within a search window. The method further comprises controlling a stimulus generator to generate a stimulus such that it is processed by the subject in an event time window comprising the future point in time.

Stimulating slow oscillations at specific phases is known to enhance deep sleep and therewith glymphatic activity. Increased glymphatic activity is known to result in increased brain waste clearance. Hence, the method may also be considered a method to stimulate brain waste clearance. The method may also be considered a method for treatment of glymphatic activity insufficiency.

The slow-oscillation range may be defined broadly within the range 0.25-4.5 Hz, but is typically defined more narrowly, e.g., as the range 0.25-4 Hz, the range 0.5-2 Hz, or as the range 0.5-1.5 Hz.

As used herein, a fitting parameter refers to a parameter of the fitted function whose value is determined by execution of the fitting algorithm.

The fitted periodic function may be a continuous trigonometric function, e.g., a sine-based function such as f(t)=A sin(v t−φ)+C, wherein t is an independent parameter representing time and wherein A, v, φ, and C are fitting parameters. A may be referred to as the amplitude, v may be referred to as the frequency, φ may be referred to as the phase offset, and C may be referred to as the constant offset.

By using a periodic function where the frequency (or equivalently, the period) is a fitting parameter, brain activity with a frequency anywhere in the slow-oscillation range may be fitted. Similarly, by using a periodic function where the amplitude is a fitting parameter, brain activity with any amplitude may be fitted. Similarly, by using a periodic function where the phase offset is a fitting parameter, brain oscillations at any phase offset may be fitted. Thus, the described method is very flexible to adapt to, e.g., temporal and/or interindividual variance in the signal. This is especially relevant when the subject has irregular and/or low-amplitude brain activity, as may be the case in, in particular, the sick and elderly. That is, the described method works reliably also in typical patients in need of stimulation of glymphatic activity (and not just in young and healthy volunteers, whose brain activity tends to show a stronger and easier to analyse signal).

The predetermined pattern can comprise (or be), e.g., a predetermined phase or predetermined phase range of the fitted periodic function; a more generically defined feature such as a peak, a trough (or negative peak), a rising slope, a falling slope, et cetera; or a predetermined phase offset with respect to such a feature. Additionally or alternatively, the predetermined pattern may comprise a predetermined amplitude or predetermined amplitude range of the fitted periodic function. As the fitted periodic function has been fitted to the data representative of brain activity of the subject and has been determined to have a frequency is in a slow-oscillation range, the predetermined pattern of the fitted periodic function may be assumed to correspond to an equal or similar pattern in the slow oscillation.

The method effectively defines a closed loop, comprising the stages of: exhibiting brain activity by the subject, measuring the brain activity, processing the measured activity (essentially in real-time), determining a point in time to provide a stimulus, providing the stimulus to the subject, and the subject exhibiting (altered) brain activity. Each of these steps introduces a certain amount of lag. Hence, a system that would attempt to instantaneously affect brain activity when a predetermined pattern in the brain activity is observed, would by necessity be too late. This is why the prediction of the future point in time at which the predetermined pattern will occur, increases the accuracy of the provision of the stimulus to coincide with a predetermined brain activity pattern.

It is noted that the processing of the stimulus by the subject may be unconscious, in particular when the subject is sleeping; however, the processing can generally be observed by a change in the detected brain activity. A stimulus that does not result in a detectable change in brain activity may be considered indiscernible.

The future point in time at which slow-wave brain activity of the subject exhibits the predetermined pattern may also be referred to as the “event time”.

The event time window may have a predetermined size. The predetermined size of the event time window may be very small, e.g., at most 2 ms, or at most 1 ms.

In an embodiment, the predetermined pattern is a predetermined oscillatory phase. The predetermined oscillatory phase can be, e.g., a phase of 0°, 90°, 180°, or 270°, et cetera; for a sine wave, these phases correspond to, respectively, halfway a rising slope, a peak, halfway a falling slope, and a trough (or negative peak). Of course, other oscillatory phases, such as 30°, 45°, or 60°, or −30°, −45°, or −60° may similarly be targeted.

Stimuli that are not fixed in time relative to a predetermined pattern in the brain activity, including rhythmic stimuli having a fixed frequency, have been shown to have much lower or no effect on stimulation of slow oscillations and are thus not expected to improve glymphatic activity.

In an embodiment, the fitted periodic function is a sine wave, and the predetermined oscillatory phase is selected from a range between −70° and 30°, more in particular from a range between −60° and 20°, from a range between −45° and 5°, from a range between −40° and 0°, from a range between −35° and −5 °, or from a range between −30° and −10 °, for example, about −20°; these phases being defined as radial phases where a 0° phase corresponds to a midway point of an ascending slope between a trough and a subsequent peak in the fitted periodic function. It has been found that a stimulus that coincides with an oscillatory phase from the specified ranges stimulates the locus coeruleus, which in turn is known to stimulate glymphatic activity. Hence, a stimulus in such a range can be especially effective at enhancing glymphatic activity, and therewith brain waste clearance.

Thus, the predetermined pattern, and in particular the predetermined oscillatory phase, may be selected to stimulate the locus coeruleus.

In an embodiment, the method further comprises defining a fitting window. The fitting window comprises a recent time segment of the data, e.g., the most-recent time segment of the data. Thus, the fitting window is generally a moving window, which may be accumulated in a moving buffer.

In such an embodiment, executing the fitting algorithm using the data as input to obtain the fitted periodic function may comprise executing the fitting algorithm using as input (exclusively) to the data in the fitting window.

In an embodiment, the fitting window has a length corresponding to between 0.25 and 2 periods of an oscillation with a frequency in the slow-oscillation range. For example, for slow oscillations, the fitting window may have a length selected in the range 0.25-2 s, in particular in the range 0.5-1 s.

In general, because of the irregularity of brain-related signals, the length of the fitting window affects the frequency (or periodicity) of the functions that can be (reliably) fitted to the signal in the fitting window. If the fitting window is too short, the signal in the fitting window may be dominated by high-frequency components, and if the fitting window is too long, only part of the signal in the fitting window may represent slow-wave activity. Thus, on the one hand the fitting window should be long enough to reliably detect (and recognise) an oscillation in the slow-oscillation range, which suggests a minimal fitting window length of between about one half to a whole oscillation period. On the other hand, the high time variance of the brain activity signal suggests that the most reliable prediction of a continuation of the brain activity signal is obtained by using as short a fitting window length as possible.

In an embodiment, an interval between an end of the fitting window and a start of the search window is at most 100 ms. More in particular, the interval may have a duration of at least 1 ms, at least 2 ms, or at least 5 ms; and/or the interval may have a duration of at most 50 ms, at most 35 ms, or at most 20 ms, or at most 10 ms.

Generally, the interval equals (at least) the loop lag, i.e., the combined delays accrued between generation of the signal by the brain and processing of the stimulus by the brain. This way, the system can compensate for this lag, and ensure that the stimulus is processed on or close to the event time. Thus, the interval may be based on one or more of: the lag introduced by the signal processing, the lag introduced by the controlling of the stimulus generator, and the lag introduced by the time delay between the generation of the stimulus and its arrival in cortical areas where slow oscillations are generated (e.g., due to the travel time of the signal and possibly the processing time in the cerebral hierarchy). In particular, each of these steps individually may be shorter than said interval.

Consequently, the algorithm or algorithms for determining the fitted periodic function and determining the future point in time at which the brain activity of the subject exhibits the predetermined pattern, must be as fast as possible and essentially real-time. Thus, the combined run-time of the steps of: executing the fitting algorithm, determining that the frequency of the fitted periodic function is in a slow-oscillation range, predicting the future point in time at which the brain activity of the subject exhibits a predetermined pattern, and determining that the future point in time is within a search window, may be essentially real-time, more specifically less than 10 ms, less than 5 ms, less than 4 ms, less than 3 ms, less than 2 ms, or even less than 1 ms.

As noted above, an interval between the fitting window and the search window that is too short may not allow for sufficient time to provide the stimulus, taking into account the lag inherent to the system. On the other hand, if the interval is too long, the reliability of the prediction of the event time may be negatively affected.

In an embodiment, the search window has a predetermined duration, in particular a duration of at most 0.2 s, more in particular at most 0.1 s, at most 50 ms, at most 20 ms, or at most 10 ms. The (maximum) duration of the search window may be determined based on similar considerations as the length of the time interval: if the search window is too long, the decision to generate a stimulus may be taken too early, negatively affecting the reliability of the prediction of the event time. On the other hand, if the search window is too short, the event time may not be found within that window, and the opportunity to provide a stimulus may be lost.

The method as claimed in claim 1, wherein a minimal delay between subsequent stimuli is based on a period, e.g., an average period, of a signal with a frequency in the slow-oscillation range. For example, the minimal delay may be at least half of a minimal period of a slow-wave signal, or at least half of a typical period of a slow-wave signal. More specifically, the minimal delay may be, e.g. at least 0.25 s, or at least 0.5 s. This prevents stimuli being provided in too short succession when the predicted event time is slightly changed by an updated fit due to newly received brain activity data, for example.

In an embodiment, the method further comprises determining that a fit quality parameter of the fitted periodic function meets a predefined criterion. For example, a (normalized) cross correlation coefficient between the fitted periodic function and the brain activity signal may be required to exceed a predefined threshold; or a variance of the fitted periodic function relative to the brain activity signal may be required to be less than a predefined threshold; or a distance between the fitted periodic function and the brain activity signal according to a (possibly) normalised metric (e.g., based on the 1-norm, the 2-norm, or the infinity-norm) may be required to be less than a predefined threshold; or a mean squared error may be required to be less than a predefined threshold.

In an embodiment, the method further comprises determining that the fitted periodic function has a relative or absolute amplitude exceeding a predetermined threshold.

In an embodiment, the data are obtained from an electroencephalogram (EEG) device. The EEG device can be, for example, a head cap, a headband, or an in-ear EEG device. In other embodiments, the data representative of the brain activity may be acquired by other sensor devices, e.g., implanted electrodes, MRI-based devices, near infrared light and functional near-infrared spectroscopy, focussed ultrasound, and so on.

In an embodiment, the data are representative of brain activity in a frontal or central part of the brain.

In an embodiment, the data are sampled with a sample rate of at least 100 Hz, in particular at least 200 Hz or at least 500 Hz. A higher sampling rate generally provides a more veridical signal and allows for a faster reaction time, but increases the computational complexity and hence the computational lag.

In an embodiment, the step of executing the fitting algorithm is performed with a repetition rate equal to the sample rate. That way, for each newly received signal, a new fit is determined. A high rate of the fitting and predicting steps leads to a high temporal resolution of the stimulating step, allowing for a high precision in targeting the predetermined pattern.

In an embodiment, the data are raw, unfiltered data.

In an embodiment, the fitting algorithm comprises, or is, a non-linear fitting algorithm, in particular a non-linear regression algorithm. For example, the fitted periodic function may be obtained using an algorithm based on the Levenberg-Marquardt algorithm, which allows for very fast, substantially real-time fitting of the periodic function.

In an embodiment, the stimulus is an discernible, e.g. audible, acoustic stimulus. In this context, the acoustic stimulus may be considered an audible acoustic stimulus if the stimulus is perceived when the subject is awake. In particular, the acoustic stimulus may be perceived, by the subject, as a very soft, undisturbing sound during waking (as determined, e.g., during a calibration procedure prior to application of the method); in that case, a same acoustic stimulus adopted during sleep usually leads to brain responses without arousals. In other embodiments, the stimulus may be a visual stimulus (e.g., a light pulse), a tactile stimulus, an electrical stimulus, an olfactory stimulus, a vestibular stimulus, or any other kind of discernible stimulus.

In an embodiment, the method further comprises determining that the subject suffers from insufficient glymphatic activity and/or undue accumulation of brain waste substances. Such a subject would benefit particularly from a method for stimulating glymphatic activity. Determining that the subject suffers from insufficient glymphatic activity and/or undue accumulation of brain waste substances may be based, e.g., on brain imaging, e.g. using MRI or functional near-infrared spectroscopy, or on a cognitive test. For example, an increased concentration of waste metabolites (such as amyloid-β, tau, and α-synuclein) in the brain may be determined, which is indicative of insufficient glymphatic activity. Furthermore, certain neurodegenerative diseases such as Alzheimer's disease and Parkinson's disease are thought to be linked to insufficient glymphatic activity.

In an embodiment, the subject is a sleeping subject. In an embodiment, the subject is a mammal, in particular a human.

In an aspect, this disclosure relates to a system for stimulating glymphatic activity. The system comprises at least one memory storing computer-readable instructions and at least one processor, communicatively connected to the at least one memory. The at least one processor is configured to, in response to executing the computer-readable instructions, perform the steps of: receiving data representative of brain activity of the subject; executing a fitting algorithm to determine, based on the received data, a fitted periodic function, the fitted periodic function having a frequency; determining that the frequency is in a slow-oscillation range; predicting a future point in time at which slow-wave brain activity of the subject exhibits a predetermined pattern, based on the fitted periodic function; determining that the future point in time is within a search window; and sending one or more control signals to a stimulus generator, the one or more control signals configuring the stimulus generator to generate a stimulus such that it coincides with an event time window comprising the future point in time.

The system may further be configured to perform any one or more of the method steps described above.

In an embodiment, the system further comprises the stimulus generator. The stimulus generator is communicatively connected or at least communicatively connectable to the at least one processor, e.g., via a communication interface.

In an embodiment, the system further comprises a detector for detecting the brain activity of the subject. The detector may be configured to generate, in response to detecting brain activity, the data representative of brain activity of the subject. The detector is communicatively connected or at least communicatively connectable to the at least one processor, e.g., via a communication interface.

The at least one memory and the at least one processor may be embodied in a controller module. The controller module and the stimulus generator and/or the detector may be contained in a single device, e.g., a wearable device; or they may be configured as separate, communicatively connectable devices.

In an aspect, this disclosure relates to a non-transitory computer readable medium having stored thereon computer program instructions that, when executed by a processor in a computing system, configure the computing system to:
receive data representative of brain activity of a subject;
executing a fitting algorithm to determine, based on the received data, a fitted periodic function, the fitted periodic function having a frequency;
determine that the frequency is in a slow-oscillation range;
predict a future point in time at which slow-wave brain activity of the subject exhibits a predetermined pattern, based on the fitted periodic function;
determining that the future point in time is within a search window; and
control a stimulus generator to generate a stimulus such that it is processed by the subject in an event time window comprising the future point in time.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system”. Functions described in this disclosure may be implemented as an algorithm executed by a microprocessor of a computer. Furthermore, aspects of the present invention may take the form of a computer program product embodied in one or more computer readable medium(s) having computer readable program code embodied, e.g., stored, thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fibre, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fibre, cable, RF, etc., or any suitable combination of the foregoing. Computer program code for carrying out operations for aspects of the present invention may be written in any combination of one or more programming languages, including a functional or an object oriented programming language such as Java, Scala, C++, Python or the like and conventional procedural programming languages, such as the “C” programming language or similar programming languages. The program code may execute entirely on the user's computer, partly on the user's computer, as a stand-alone software package, partly on the user's computer and partly on a remote computer, or entirely on the remote computer, server or virtualized server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor, in particular a microprocessor or central processing unit (CPU), or graphics processing unit (GPU), of a general purpose computer, special purpose computer, or other programmable data processing apparatus to produce a machine, such that the instructions, which execute via the processor of the computer, other programmable data processing apparatus, or other devices create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, can be implemented by special purpose hardware-based systems that perform the specified functions or acts, or combinations of special purpose hardware and computer instructions.

### BRIEF DESCRIPTION OF THE DRAWINGS

The embodiments will be further illustrated with reference to the attached schematic drawings, in which:

FIGS. 1A and 1B schematically depict systems for stimulating glymphatic activity according to two embodiments;

FIGS. 2A and 2B depict brain activity data with a fitted periodic function, FIG. 2C depicts a reference periodic function, and FIG. 2D depicts experimental results of a method according to an embodiment;

FIG. 3 is a flowchart of a method according to an embodiment; and,

FIG. 4 depicts a block diagram illustrating an exemplary data processing system configured to perform one or more method steps according to an embodiment.

### DETAILED DESCRIPTION

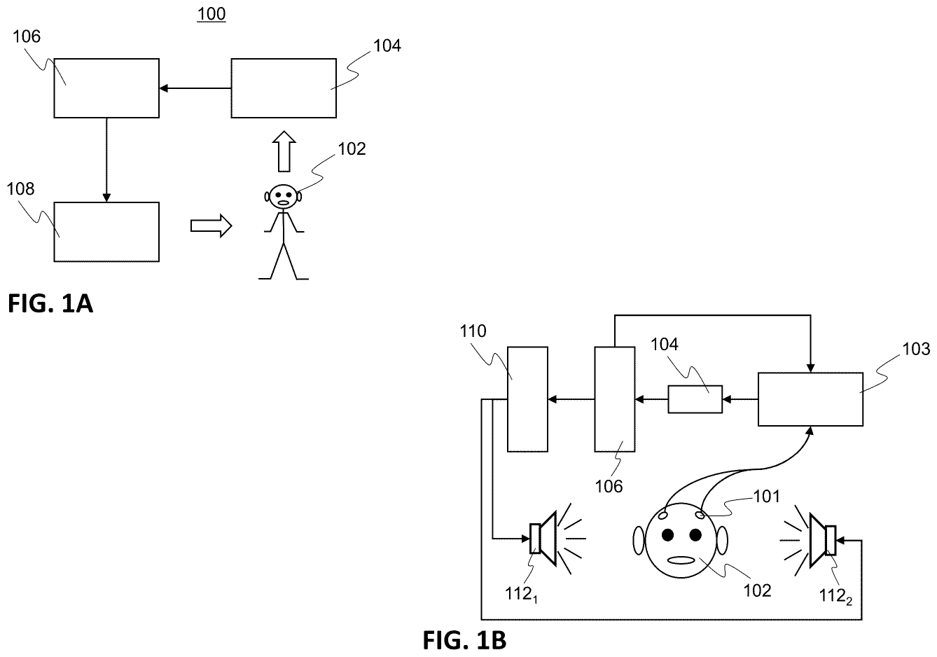

FIG. 1A schematically depicts a system for stimulating glymphatic activity according to an embodiment. The system 100 comprises a detector 104 for detecting brain activity of a subject 102, a stimulus generator 108 for providing a stimulus to the subject 102, and a controller 106 configured to receive data from the detector 104 and, in response to the received data, control the stimulus generator 108.

In the following, the system 100 will be described as it is when in use. When in use, the system is configured for stimulating glymphatic activity of the subject 102. The subject may be a sleeping subject. The subject may be a mammal, in particular a human. The subject may have been determined to suffer from insufficient glymphatic activity and/or to suffer undue accumulation of brain waste substances. Determining that the subjects suffers from insufficient glymphatic activity and/or undue accumulation of brain waste substances may be based, e.g., on brain imaging, e.g., using MRI or functional near-infrared spectroscopy, or on a cognitive test. For example, an increased concentration of waste metabolites (such as amyloid-β, tau, and α-synuclein) in the brain may be determined, which is indicative of insufficient glymphatic activity. Furthermore, certain neurodegenerative diseases such as Alzheimer's disease and Parkinson's disease are thought to be linked to insufficient glymphatic activity; hence, known tests to determine such neurodegenerative diseases, or symptoms associated with such neurodegenerative diseases, may be used to determine that the subject suffers from insufficient glymphatic activity and/or suffers undue accumulation of brain waste substances.

The detector 104 is configured to detect brain activity of the subject 102. The brain activity may be brain activity in a frontal or central part of the brain. The detector may be an electroencephalogram (EEG) device, e.g., an EEG head cap, an EEG headband, an in-ear EEG device, et cetera. In other embodiments, the data representative of the brain activity may be acquired by another sensor device, e.g., using implanted electrodes, an MRI-based device, using near-infrared light and functional near-infrared spectroscopy, using focussed ultrasound, and so on. Such detectors capable of collecting signals representative of brain activity are known in the art.

The detector 104 may be configured to sample the brain wave activity, and hence to generated the data, with a sample rate of at least 100 Hz, in particular at least 200 Hz or at least 500 Hz. A higher sampling rate generally provides a more veridical signal and allows for a faster reaction time, but may increase the computational complexity of the analysis performed by the controller 106, and may hence increase computational lag.

The detector 104 may be configured to generate, in response to detecting brain activity, data representative of brain activity of the subject 102. The detector is communicatively connected or at least communicatively connectable to the controller 106, e.g., via a communication interface. This connection may be wired or wireless, using known communication protocols.

The controller 106 comprises at least one memory storing computer-readable instructions and at least one processor, communicatively connected to the at least one memory. Executing the computer-readable instructions configures the at least one processor to perform a method as described herein.

The controller 106 can be implemented as, e.g., a generic data processing system such as personal computer, laptop, tablet, smart phone, smart watch or other smart wearable device. Alternatively, the controller 106 may be implemented as a dedicated device, e.g., an integrated circuit, FPGA, et cetera.

In particular, the at least one processor may be configured to receive, from the detector 104, data representative of the brain activity of the subject 102. The at least one processor may then execute a fitting algorithm, using the data as input, to obtain a fitted periodic function. The data used as input may be raw, unfiltered data; alternatively, the data may be filtered or otherwise pre-processed data. In the latter case, the filtering or otherwise pre-processing may be performed by the detector 104, by the controller 106, or by some other (intermediate) device.

The fitted periodic function has a frequency, which frequency is generally one of one or more fitting parameters which are adjusted by the fitting algorithm to obtain the fitted periodic function. The fitting algorithm may be essentially real-time; e.g., the fitting algorithm may determine a fitted periodic function within 20 ms from receiving the data, or even withing 15 ms, 10 ms, 5 ms, 4 ms, 3 ms, 2 ms, 1 ms, or 0.5 ms from receiving the data. The fitting algorithm may be, or at least comprise, a non-linear fitting algorithm, such as non-linear regression algorithm. For example, the fitting algorithm may be based on the Levenberg-Marquardt algorithm.

The at least one processor may be configured to execute the fitting algorithm with a repetition rate of at least 100 Hz, in particular at least 200 Hz or at least 500 Hz. More specifically, the repetition rate may be equal to the sample rate of the detector 104. That way, for each sample (or each newly received data point), a new fitted periodic function is determined. A high rate of the fitting and predicting steps leads to a high temporal resolution of the stimulating step (described below), allowing for a high precision in targeting a desired pattern in the brain activity signal.

The fitted periodic function may be a continuous trigonometric function, e.g., a sine-based function such as f(t)=A sin(v t−φ)+C, wherein t is an independent parameter representing time and wherein A, v, φ, and C are fitting parameters. A may be referred to as an amplitude of the fitted periodic function, v may be referred to as a frequency of the fitted periodic function, φ may be referred to as a phase offset of the fitted periodic function, and C may be referred to as a constant offset of the fitted periodic function.

The fitting algorithm may use as input the brain activity data within a fitting window with a predefined length. The fitting window may sometimes also be referred to as a calculus window. The fitting window generally comprises a recent time segment of the data, e.g., the most-recent time segment of the data. Thus, the fitting window is generally a moving window, which may be accumulated in a moving buffer. The fitting window may have a length corresponding to between 0.25 and 2 periods of an oscillation with a frequency in the slow-oscillation range. For example, for slow oscillations, the fitting window may have a length selected in the range 0.25-2 s, in particular in the range 0.5-1 s.

The at least one processor may further be configured to determine that the frequency (of the fitted periodic function) is in the slow-oscillation range. The slow-oscillation range may be defined broadly within the range 0.25-4.5 Hz, but may also be defined more narrowly, e.g., as the range 0.25-4 Hz, the range 0.5-2 Hz, or as the range 0.5-1.5 Hz. As the fitted periodic function has been fitted to the brain activity data, the fitted periodic function having a frequency in the slow-oscillation range is indicative for the brain activity exhibiting slow oscillations. The brain activity exhibiting slow oscillations may also be referred to as slow-wave activity (SWA), or, if the subject 102 is asleep, as slow-wave sleep (SWS).

The at least one processor may further be configured to predict, based on the fitted periodic function, a future point in time at which the brain activity of the subject exhibits a predetermined pattern. The future point in time may also be referred to as the event time. The predetermined pattern can be a predetermined oscillatory phase. The predetermined oscillatory phase can be, e.g., −20°(or equivalently, 340°) of a sine function; i.e., roughly in the second quartile of a phase difference between a trough and a subsequent peak.

More in general, the predetermined oscillatory phase may be selected from a range between −70° and 30°, more in particular from a range between −60° and 20°, from a range between −45° and 5°, from a range between −40° and 0°, from a range between −35° and −5°, or from a range between −30° and −10°, as defined relative to a sine wave.

The future point in time may be limited in time to be within a predefined search window. The search window may be defined relative to ‘now’, i.e., relative to a clock time of the controller 106 at the moment of performing the step of predicting the future point in time. Additionally or alternatively, the search window may be defined relative to (an end time of) the fitting window and/or a time at which a most recent data point of the data representative of the brain activity of the subject 102 was collected or received.

In particular, a starting point of the search window may be defined such that an interval between an end of the fitting window (or reception or collection of the most recent data point) and a start of the search window is at least the length of the loop lag. The loop lag will be described in more detail below, but generally refers to the combined delays that occur between generation of the signal by the brain of the subject 102 and processing by the subject of the stimulus generated by the system 100. In particular, the interval may have a duration of at most 100 ms. More in particular, the interval may have a duration of at least 2 ms, at least 5 ms, or at least 10 ms; and the interval may have a duration of at most 50 ms, at most 35 ms, or at most 20 ms.

In general, the interval may be based on one or more of: the lag introduced by the signal processing, the lag introduced by the controlling of the stimulus generator, and the lag introduced by the time delay between the generation of the stimulus and its arrival in cortical areas where slow oscillations are generated (e.g., due to the travel time of the signal and the processing time in the cerebral hierarchy). In particular, each of these steps individually may be shorter than said interval.

The search window may have a predetermined duration, in particular a duration of at most 0.2 s, more in particular at most 0.1 s, at most 50 ms, at most 20 ms, or at most 10 ms. The (maximum) duration of the search window may be determined based on similar considerations as the length of the time interval between the end of the fitting window and/or the collection or reception of the most recent data point described above: if the search window is too long, the decision to generate a stimulus may be taken too early, negatively affecting the reliability of the prediction of the event time. On the other hand, if the search window is too short, the event time may not be found within that window, and the opportunity to provide a stimulus may be lost.

The performing of the prediction, by the at least one processor, of the event time may be conditional on the fitted periodic function meeting one or more criteria. Hence, the at least one processor may be configured to determine whether the fitted periodic function meets a predefined criterion. This criterion may be indicative of a quality of the fit, i.e., a measure how well the fitted periodic function fits the data representative of the brain activity. For example, the at least one processor may be configured to determine whether a (possibly normalized) cross correlation coefficient between the fitted periodic function and the brain activity signal exceeds a predefined (first) threshold; and/or to determine whether a variance of the fitted periodic function relative to the brain activity signal does not exceed a predefined (second) threshold; and/or to determine whether a distance between the fitted periodic function and the brain activity signal according to a (possibly normalised) metric (e.g., based on the 1-norm, the 2-norm, or the infinity-norm) does not exceed a predefined (third) threshold; and/or to determine whether a mean squared error does not exceed a predefined (fourth) threshold. Additionally or alternatively, the criterion may be based solely on (fitting parameters of) the fitted periodic function; for example, the at least one processor may be configured to determine whether the fitted periodic function has a relative or absolute amplitude exceeding a predetermined (fifth) threshold.

The at least one processor may further be configured to control the stimulus generator 108 to generate a stimulus such that it is processed by the subject in an event time window comprising the future point in time. The stimulus generator is communicatively connected or at least communicatively connectable to the at least one processor, e.g., via a communication interface. This connection may be wired or wireless, using known communication protocols.

The stimulus may be generated such that its arrival coincides with the predicted predetermined pattern. To this end, the stimulus may be generated a short amount of time (e.g., on the order of one to several milliseconds) before the event time, based on, e.g., the time required for the stimulus to travel from the stimulus generator 108 to the subject 102.

The stimulus may be an (audible) acoustic stimulus. In that case, the stimulus generator may comprise one or more loudspeakers, or a different kind of transducer. In other embodiments, the stimulus may be a visual stimulus (e.g., a light pulse), a tactile stimulus, an electrical stimulus, an olfactory stimulus, a vestibular stimulus, or any other kind of discernible stimulus. Correspondingly, the stimulus generator 108 may comprise one or more of:
a light source, which, when in use, is arranged to illuminate the subject, in particular the eyes of the subject 102;
an actuator which, when in use, is arranged to move an object against the subject 102;
one or more electrodes which, when in use, are arranged to apply an electric signal to the subject 102; or
an actuator which, when in use, is arranged to move an object, e.g., a bed or mattress, on which the subject 102 is positioned.

The at least one processor of the controller 106 may be configured to ensure that subsequent stimuli are generated with a minimal delay between the subsequent stimuli. The minimal delay between the subsequent stimuli may be based on an (average or typical) period of a signal with a frequency in the slow-oscillation range. For example, the minimal delay may be at least half of a minimal period of a slow-oscillation signal, or at least half of a typical period of a slow-wave signal. More specifically, the minimal delay may be, e.g. at least 0.25 s, or at least 0.5 s.

Although depicted here as three different entities, the controller 106 and the stimulus generator 108 and/or the detector 104 may be contained in a single device, e.g., a wearable device. Alternatively, they may be configured as two, three, or more separate, communicatively connectable devices. For example, the system may be implemented using hardware equal to or similar to that used in the commercially available DREEM 2 headband, combining the detector, controller, and stimulus generator in a single wearable device.

As is shown by the arrows in FIG. 1A, the system 100 together with the subject 102 effectively defines a closed loop. This closed loop comprising the stages of:
the subject exhibiting brain activity,
measuring the brain activity to obtain data representative of brain activity of the subject,
processing the obtained data (essentially in real-time), including determining a fitted periodic function that fits the obtained data and determining that the fitted periodic function has a frequency in the slow-oscillation range,
determining a point in time to provide a stimulus, by predicting a future point in time at which the brain activity of the subject exhibits a predetermined pattern and determining that the future point in time is within a search window,
providing the stimulus to the subject, and
the subject exhibiting (altered) brain activity.

Each of these steps introduces a certain amount of lag, which may range from microseconds to milliseconds, which can be compensated for by the described system.

Thus, the prediction of the future point in time at which the predetermined pattern will occur, ensures that the stimulus is provided to coincide with a predetermined brain activity pattern with high accuracy.

FIG. 1B schematically shows a specific embodiment of a system as described herein. In FIG. 1B, the stimulus generator comprises a sound amplifier 110 and set of speakers 1121,2 for providing a sound signal as stimulus. In this example, the brain-related signal from subject 102 is first amplified using an amplifier 103 and then transmitted using a glass fiber coupling to a data processor 104 which records an EEG signal. The EEG signal comprises data representative of brain activity of the subject. The data processor 104 is coupled to a computer system 106, here via a wired coupling over data cable. On the computer system 106, a computer program runs which, essentially real-time, receives (e.g., retrieves) the EEG signal, and executes a fitting algorithm to determine, based on the EEG signal, a fitted periodic function having a frequency. The computer program determines that the frequency is in a slow-oscillation range, and predicts, based on the fitted periodic function, a future point in time at which the brain activity of the subject exhibits a predetermined pattern. The computer system then activates the stimulus generator for providing a stimulus to the subject 102 such that it is processed by the subject in an event time window comprising the future point in time.

An example of the equipment and software which can be used in the current assembly is summed below.
64-channel EEG cap 101 WaveGuard original (ANT-neuro)
72-channel Refa Amplifier 103 (TMSi)
customized version of PolyBench software (TMSi; adapted by UvA, with working data stream)
EventIDE software (Okazolab), a software package for experiment control and stimulus presentation, which hosts an algorithm for real-time EEG analysis.
Generic equipment/software:
EEG recording data processor 104 with:
Intel Core i5-2500 CPU @ 3.30 GHz processor
8.00 GB RAM intern memory
465 GB SSD extra hard disk
2x network card
Computer system 106 for running the computer program:
Intel Core i5-4690 CPU @ 3.50 GHz processor
16.0 GB RAM intern memory
2x network card
Sound Blaster Z-series sound card 110 LPT port (parallel/printer port)
Geforce GTX 750 video card
Further equipment:
Cross-over network cable cat. 6, max. 1 m length
2x Creative speakers 112_{1,2} Gigaworks T20 series II

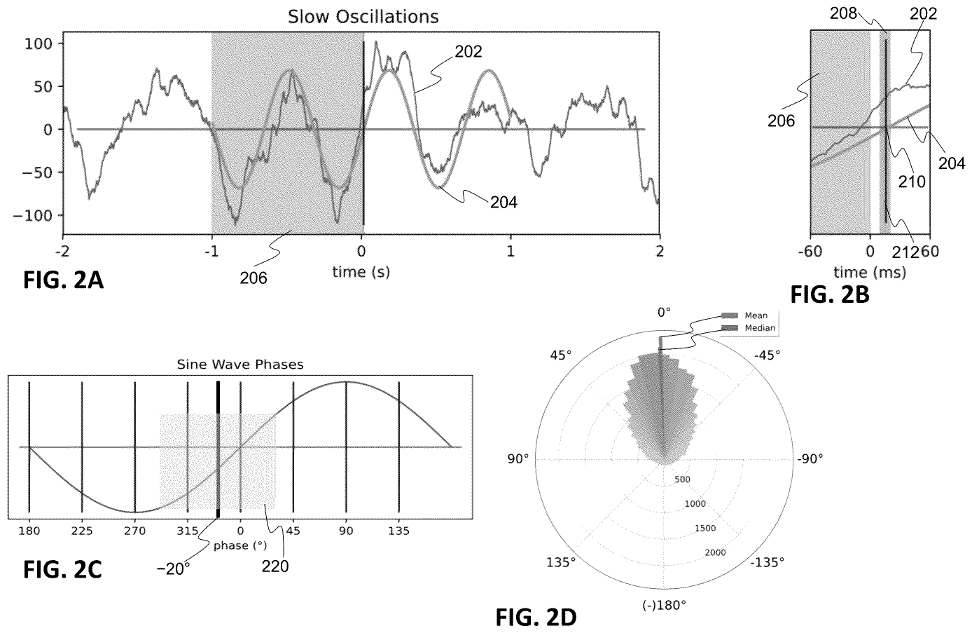

FIGS. 2A and 2B depict data representative of brain activity 202 of a subject, in this case a sleeping human subject, and a fitted periodic function 204 that has been fitted to said data using an algorithm as defined herein. In this graph, time t=0 (also referred to ‘now’) represents the time the most recent data point of the data representative of the brain activity was received. The shaded area 206 represents the fitting window, in this case extending 1.0 s back from now. In the depicted example, the fitted periodic function has a frequency of about 1 Hz (and hence a period of about 1 s), and is therefore indicative of the presence of slow-wave activity (and in this case, slow-wave sleep) in the brain of the subject.

FIG. 2B is a close-up of FIG. 2A, around time t=0. The dark shaded area 208 represents the search window, which in this example has a size of 12 ms. The interval between the end of the fitting window and the start of the search window is 10 ms. The algorithm for determining a fitted periodic function and determining a point in time at which a predetermined pattern occurs, runs within this interval (typically in the very first part of this interval). In the depicted example, the predetermined pattern 210 is an oscillatory phase of 0°, as defined for a sine wave (see also FIG. 2C). The future time (with respect to ‘now’) is indicated with a vertical black bar 212.

FIG. 2C depicts a reference periodic function, in particular a sine wave. Different phases defined relative to the sine wave are shown. In particular, an oscillatory phase of −20° (or 340°) has been marked by a thick vertical bar. A shaded region 220 of ±50°, extending from −70° to 30°, is also shown.

During deep sleep, two mechanisms contribute to enhanced brain waste clearance: first, synchronized neuronal activity during sleep slow waves generates large-amplitude rhythmic ionic waves in the interstitial fluid (ISF), which may facilitate cerebrospinal fluid—interstitial compartment perfusion and movement of metabolic waste within the interstitial fluid (Jiang-Xie et al., ‘Neuronal dynamics direct cerebrospinal fluid perfusion and brain clearance’ Nature 627:157-164 (February 2024), pages 157-164). Second, norepinephrine excretion by the locus coeruleus show rhythmic oscillations, phase-locked to sleep slow waves. This rhythmic norepinephrine pulses, directly drive the arterial pulsations underlying glymphatic activity (Hauglund et al., ‘Norepinephrine-mediated slow vasomotion drives glymphatic clearance during sleep’Cell 188:3 (February 2025), pages 606-622).

Noradrenergic neurons of the locus coeruleus fire preferentially along the ascending edge (i.e., positive going slope of the EEG slow oscillation, correlating with down-to-up state transitions), as shown by Eschenko et al., ‘Noradrenergic neurons of the locus coeruleus are phase locked to cortical up-down states during sleep’, Cerebral Cortex 22:2, (February 2012) pages 426-435. The distribution of preferred phase for all significantly modulated locus coeruleus units was concentrated within the range of −70° to 30° (shaded region 220 in FIG. 2C), with (circular) population mean at −19°. This range corresponds to the ‘lower portion’ of the ascending (depolarizing) phase of the slow wave.

Therefore, the predetermined pattern may be a predetermined oscillatory phase of −19° or −20 °, or more broadly predetermined oscillatory phase in the range of −70° to 30°, as defined relative to a sine wave. Stimulating brain activity at such a oscillatory phase can stimulate the locus coeruleus, and thereby enhance brain waste clearance.

FIG. 2D depicts experimental results of a method according to an embodiment. The results show that the methods described herein have a high accuracy in targeting a predefined oscillatory phase. The offset from the target phase is 1.85°±49.16° (mean±SD), based on 36693 stimulus onset markers. In these experiments, the averaged inter-stimulus-interval was 1.82 +1.59 seconds (mean±SD). Given that slow oscillations have a maximum duration of about two seconds (0.5 Hz), this indicates that most naturally occurring slow oscillations were targeted.

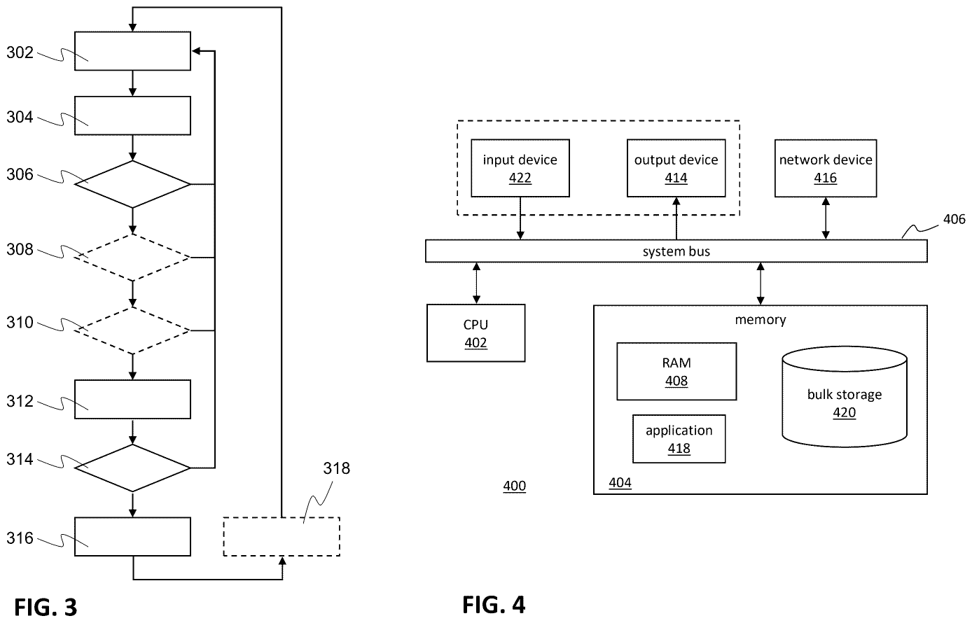

FIG. 3 is a flowchart of a method according to an embodiment. The method may be performed, for example, by the controller 106 as described above with reference to FIG. 1A.

A first step 302 comprises receiving data representative of brain activity of the subject. The data may be representative of brain activity in a frontal or central part of the brain. The data may be received from an EEG device or another device suitable for monitoring brain activity. The data may have a sample rate in a range of about 100-1000 Hz.

The subject may be a mammal, e.g., a human. The subject may be a sleeping subject. In an optional preliminary step, the subject may have been determined to suffer from insufficient glymphatic activity or from undue accumulation of brain waste substances.

A step 304 comprises executing a fitting algorithm to determine, based on the received data, a fitted periodic function, the fitted periodic function having a frequency. The fitting algorithm may be a non-linear algorithm, e.g., a non-linear regression algorithm. The fitting algorithm may run essentially in real time; e.g., the run time of the fitting algorithm may be substantially less than a time interval between two samples in the received data. The data used as input by the fitting algorithm may be raw, unfiltered data. The fitting algorithm may use, as input, data from a fitting window comprising a recent, e.g. a most recent, time segment of the data. The fitting window may have a length between, e.g., 0.25-2 periods of an oscillation with a frequency in the slow-oscillation range.

A step 306 comprises determining that the frequency of the fitted periodic function is a frequency in the slow-oscillation range. If the frequency of the fitted periodic function is not a frequency in the slow-oscillation range, the current iteration of the method may abort and the method may start a new iteration, returning to step 302.

An optional step 308 comprises determining that a fit quality parameter of the fitted periodic function meets a predefined criterion. The fit quality parameter may be determined based on a comparison of the received data and the fitted periodic function. If the fit quality parameter of the fitted periodic function does not meet the predefined criterion, the current iteration of the method may abort and the method may start a new iteration, returning to step 302.

An optional step 310 comprises determining that the fitted periodic function has a relative or absolute amplitude exceeding a predetermined threshold. If the fitted periodic function does not have a relative or absolute amplitude exceeding the predetermined threshold, the current iteration of the method may abort and the method may start a new iteration, returning to step 302.

A step 312 comprises predicting, based on the fitted periodic function, a future point in time at which the brain activity of the subject exhibits a predetermined pattern. The term predicting may be understood as forecasting, i.e., the prediction is data-driven and is statistically known to how a certain accuracy. The predetermined pattern may be a predetermined oscillatory phase. The predetermined pattern may be selected to stimulate the locus coeruleus. The predetermined oscillatory phase may be selected from a range between −70° and 30°, e.g., as approximately −20°, as defined relative to a sine wave.

A step 314 comprises determining that the future point in time is within a search window. The search window may have a duration of at most 0.2 s. A start point of the search window may be defined such that an interval between an end point of the fitting window and the start point of the search window is less than 100 ms, e.g., less than 50 ms. Additionally or alternatively, the start point of the search window may be defined such that an interval between a collection time or a reception time of a most recent data point from the received data and the start point of the search window is less than 100 ms, e.g., less than 50 ms. If the future point in time is not within the search window, the current iteration of the method may abort and the method may start a new iteration, returning to step 302.

A step 316 comprises controlling a stimulus generator to generate a stimulus such that it is processed by the subject in an event time window comprising the future point in time. The stimulus may be a discernible acoustic stimulus.

An optional step 318 comprises waiting, after a stimulus has been generated (and provided to the subject), for a predetermined amount of time. The predetermined amount of time may be based on a (desired) minimal delay between subsequent stimuli. The minimal delay between subsequent stimuli may be based on a period (e.g., an average or typical period) of a signal with a frequency in the slow-oscillation range. After waiting the predetermined amount of time, the method may restart, beginning again at step 302. Alternatively, the method could comprise a step, prior to step 316, that comprises determining that the predetermined amount of time has passed since the last stimulus was given and, if the predetermined amount of time has not passed since the last stimulus was given, aborting the current iteration of the method and starting a new iteration, returning to step 302.

In a typical application, the method is run continuously, allowing frequent stimulation of the subject, in particular during NREM3 sleep phases. NREM3 sleep is also known as deep sleep, or slow-wave sleep (SWS). For example, it has been found that using the method described above, during artifact-free NREM3 sleep, typically between 14 and 32 stimuli are generated each minute to stimulate slow oscillations with a frequency between 0.5-1.5 Hz. These data cover both disturbed and healthy sleepers, with disturbed sleepers being, on average, a bit lower in this distribution than healthy sleepers. These data were obtained stimulation over an EEG headband (rather than state of the art lab equipment), which in itself makes for a weaker signal (i.e., with a lower amplitude, and lower signal-to-noise ratio) compared to lab equipment. Thus, these results are indicative of what could be achieved using a commercial home-use device.

Thus, in some embodiments, the method steps 302-316 (or 302-318, depending on the implementation) are repeated substantially continuously; e.g., the method may start at step 302 at least 10 times per second, more typically at least 50 times per second or at least 100 times per second. The continuous running of the method may be maintained for a substantial amount of time, e.g., at least one hour, but more typically an entire night, or an entire time interval between a person going to bed or starting to sleep, and the person getting up or waking up. While the method is being continuously repeated, the method may further comprise providing at least 1, in particular at least 2, at least 3, at least 4, at least 5, at least 6, at least 7, at least 8, at least 3, or at least 4 stimuli per minute of NREM3 sleep of the subject.

FIG. 4 depicts a block diagram illustrating an exemplary data processing system that may perform the method as described with reference to FIG. 3.

As shown in FIG. 4, the data processing system 400 may include at least one processor 402 coupled to memory elements 404 through a system bus 406. As such, the data processing system may store program code within memory elements 404. Further, the processor 402 may execute the program code accessed from the memory elements 404 via a system bus 406. In one aspect, the data processing system may be implemented as a computer that is suitable for storing and/or executing program code. It should be appreciated, however, that the data processing system 400 may be implemented in the form of any system including a processor and a memory that is capable of performing the functions described within this specification. The data processing system may be an Internet/cloud server, for example.

The memory elements 404 may include one or more physical memory devices such as, for example, local memory 408 and one or more bulk storage devices 410. The local memory may refer to random access memory or other non-persistent memory device(s) generally used during actual execution of the program code. A bulk storage device may be implemented as a hard drive or other persistent data storage device. The processing system 400 may also include one or more cache memories (not shown) that provide temporary storage of at least some program code in order to reduce the quantity of times program code must be retrieved from the bulk storage device 410 during execution. The processing system 400 may also be able to use memory elements of another processing system, e.g. if the processing system 400 is part of a cloud-computing platform.

Input/output (I/O) devices depicted as an input device 412 and an output device 414 optionally can be coupled to the data processing system. Examples of input devices may include, but are not limited to, a keyboard, a pointing device such as a mouse, a microphone (e.g. for voice and/or speech recognition), or the like. Examples of output devices may include, but are not limited to, a monitor or a display, speakers, or the like. Input and/or output devices may be coupled to the data processing system either directly or through intervening I/O controllers.

In an embodiment, the input and the output devices may be implemented as a combined input/output device (illustrated in FIG. 4 with a dashed line surrounding the input device 412 and the output device 414). An example of such a combined device is a touch sensitive display, also sometimes referred to as a “touch screen display” or simply “touch screen”. In such an embodiment, input to the device may be provided by a movement of a physical object, such as e.g. a stylus or a finger of a user, on or near the touch screen display.

A network adapter 416 may also be coupled to the data processing system to enable it to become coupled to other systems, computer systems, remote network devices, and/or remote storage devices through intervening private or public networks. The network adapter may comprise a data receiver for receiving data that is transmitted by said systems, devices and/or networks to the data processing system 400, and a data transmitter for transmitting data from the data processing system 400 to said systems, devices and/or networks. Modems, cable modems, and Ethernet cards are examples of different types of network adapter that may be used with the data processing system 400.

As pictured in FIG. 4, the memory elements 404 may store an application 418. In various embodiments, the application 418 may be stored in the local memory 408, the one or more bulk storage devices 410, or separate from the local memory and the bulk storage devices. It should be appreciated that the data processing system 400 may further execute an operating system (not shown in FIG. 4) that can facilitate execution of the application 418. The application 418, being implemented in the form of executable program code, can be executed by the data processing system 400, e.g., by the processor 402. Responsive to executing the application, the data processing system 400 may be configured to perform one or more operations or method steps described herein.

Various embodiments of the invention may be implemented as a program product for use with a computer system, where the program(s) of the program product define functions of the embodiments (including the methods described herein). In one embodiment, the program(s) can be contained on a variety of non-transitory computer-readable storage media, where, as used herein, the expression “non-transitory computer readable storage media” comprises all computer-readable media, with the sole exception being a transitory, propagating signal. In another embodiment, the program(s) can be contained on a variety of transitory computer-readable storage media. Illustrative computer-readable storage media include, but are not limited to: (i) non-writable storage media (e.g., read-only memory devices within a computer such as CD-ROM disks readable by a CD-ROM drive, ROM chips or any type of solid-state non-volatile semiconductor memory) on which information is permanently stored; and (ii) writable storage media (e.g., flash memory, floppy disks within a diskette drive or hard-disk drive or any type of solid-state random-access semiconductor memory) on which alterable information is stored. The computer program may be run on the processor 402 described herein.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the embodiments in the form disclosed. Many modifications and variations will be apparent to those of ordinary skill in the art without departing from the scope and spirit of the invention. The embodiments were chosen and described in order to best explain the principles and the practical application, and to enable others of ordinary skill in the art to understand the various embodiments with various modifications as are suited to the particular use contemplated.

1. A method for stimulating glymphatic activity in a subject, the method comprising:
receiving data representative of brain activity of the subject;
executing a fitting algorithm to determine, based on the received data, a fitted periodic function, the fitted periodic function having a frequency;
determining that the frequency is in a slow-oscillation range;
predicting a future point in time at which the brain activity of the subject exhibits a predetermined pattern, based on the fitted periodic function;
determining that the future point in time is within a search window; and
controlling a stimulus generator to generate a stimulus such that it is processed by the subject in an event time window comprising the future point in time.

2. The method as claimed in claim 1, wherein the predetermined pattern is a predetermined oscillatory phase.

3. The method as claimed in claim 2, wherein the fitted periodic function is a sine wave and wherein the predetermined oscillatory phase is selected from a range between −70° and 30°.

4. The method as claimed in claim 1, further comprising defining a fitting window, the fitting window comprising a recent time segment of the data,
wherein executing the fitting algorithm to determine, based on the received data, a fitted periodic function comprises executing the fitting algorithm using as input the data in the fitting window.

5. The method as claimed in claim 4, wherein the fitting window has a length corresponding to between 0.25 and 2 periods of an oscillation with a frequency is in the slow-oscillation range.

6. The method as claimed in claim 4, wherein an interval between an end of the fitting window and a start of the search window is at most 100 ms.

7. The method as claimed in claim 1, wherein the search window has a duration of at most 0.2 s.

8. The method as claimed in claim 1, wherein a minimal delay between subsequent stimuli is based on a period of a signal with a frequency in the slow-oscillation range.

9. The method as claimed in claim 1, further comprising determining that a fit quality parameter of the fitted periodic function meets a predefined criterion.

10. The method as claimed in claim 1, further comprising determining that the fitted periodic function has a relative or absolute amplitude exceeding a predetermined threshold.

11. The method as claimed in claim 1, wherein the data are obtained from an EEG device.

12. The method as claimed in claim 1, wherein the data are representative of brain activity in a frontal or central part of the brain.

13. The method as claimed in claim 1, wherein the data are sampled with a sample rate of at least 100 Hz, and wherein the step of executing the fitting algorithm is performed with a repetition rate equal to the sample rate.

14. The method as claimed in claim 1, wherein the data are raw, unfiltered data.

15. The method as claimed in claim 1, wherein the fitting algorithm comprises a non-linear regression algorithm.

16. The method as claimed in claim 1, wherein the stimulus is a discernible acoustic stimulus.

17. The method as claimed in claim 1, further comprising determining that the subject suffers from insufficient glymphatic activity or undue accumulation of brain waste substances.

18. The method as claimed in claim 1, wherein the subject is a sleeping mammal.

19. A system comprising:
at least one memory storing computer-readable instructions; and
at least one processor, communicatively connected to the at least one memory;
wherein the at least one processor is configured to, in response to executing the computer-readable instructions, perform the steps of:
receiving data representative of brain activity of the subject;
executing a fitting algorithm to determine, based on the received data, a fitted periodic function, the fitted periodic function having a frequency;
determining that the frequency is in a slow-oscillation range;
based on the fitted periodic function, predicting a future point in time at which slow-wave brain activity of the subject exhibits a predetermined pattern;
determining that the future point in time is within a search window; and
sending one or more control signals to a stimulus generator, the one or more control signals configuring the stimulus generator to generate a stimulus such that it is processed by the subject in an event time window comprising the future point in time.

20. The system as claimed in claim 19, further comprising the stimulus generator.

21. The system as claimed in claim 19, further comprising a detector for detecting the brain activity of the subject.

22. A non-transitory computer readable medium having stored thereon computer program instructions that, when executed by a processor in a computing device, configure the computing device to:
receive data representative of brain activity of a subject;
fit a periodic function to the data, the fitted periodic function having a frequency;
determine that the frequency is in a slow-oscillation range;
predict a future point in time at which slow-wave brain activity of the subject exhibits a predetermined pattern, based on the fitted periodic function;
determining that the future point in time is within a search window; and
control a stimulus generator to generate a stimulus such that it is processed by the subject in an event time window comprising the future point in time.
